(19) United States
(12) Patent Application Publication
(10) Pub. No.: US 2009/0204045 A1
(43) Pub. Date: Aug. 13, 2009

(54) Corrective Device for Ingrown Toenail and Incurvated Toenail

(76) Inventor: Jae Young Kim, Seoul (KR)

Correspondence Address: PRICE HENEVELD COOPER DEWITT & LITTON, LLP, 695 KENMOOR, S.E., P O BOX 2567, GRAND RAPIDS, MI, 49501, US

(21) Appl. No.: 12/094,640

(22) PCT Filed: Sep. 28, 2007
(86) PCT No.: PCT/KR07/04762

§ 371 Date: Oct. 3, 2008

(30) Foreign Application Priority Data
Sep. 28, 2006 (KR) 10-2006-0094451

(51) Int. Cl. A61F 5/01

(52) U.S. Cl. 602/31

(57) ABSTRACT

Provided is a corrective device for ingrown toenails and incurvated toenails. The corrective device includes a central member made of a shape-memory alloy; and prefabricated toenail side-engaging parts, each being attached to both ends of the central member. The corrective device of the present invention has advantages such as provision of strong corrective force over the entire toenail, convenient use, suitability for long-term use, straight growth of a toenail that will grow after a surgical treatment of the ingrown toenail, and straightening of a curved toenail.

### TECHNICAL FIELD

The present invention relates to a corrective device for ingrown toenails and incurvated toenails. More specifically, the present invention relates to a corrective device for treatment of ingrown toenails and incurvated toenails, which has strong corrective force throughout the entire toenail, can be conveniently used, is suitable for long-term use, and directs the toenail that will grow after a surgical treatment of the ingrown toenail to grow straight.

### BACKGROUND ART

As shown in FIG. 1, a human toenail 10 serves to protect a tiptoe and toe bones (phalanges), and is made up of three parts, a nail body 1, a nail root 3 and a free edge 5. The nail root 3 is the proximal part of the nail from which the nail begins to grow. The nail root 3 is at the base of the nail and is embedded underneath the skin. The free edge 5 is the extension of the nail plate that grows beyond the edge of the toe.

The skin surrounding such a toenail consists of cuticle 7 preventing intrusion of pathogenic bacteria, nailwalls 9 corresponding to the skin on both lateral sides of the toenail, and nailgrooves 11 corresponding to the groove between the nailwall 9 and the toenail 10.

As used herein, the term “ingrown toenail” refers to a condition of toenail deformity in which the toenail curves downward and grows into the nailgrooves 11, whereas the term “incurvated toenail” refers to a condition of the toenail deformity in which the nail body 1 grows into both nailgrooves 11 to penetrate into the subcutaneous tissue and thereby grows along the nailwall 9. Causes responsible for the occurrence of the incurvated toenail may include tinea unguium (onychomycosis), deformity or structural abnormality of phalanges, unsanitary environment, and improper trimming of toenails, in conjunction with an additional cause such as wearing of pointed-toe shoes or high-heeled shoes that imposes pressure on the toenails. The ingrown toenail and incurvated toenail cause severe pain upon wearing of the shoes and are likely to result in the risk of bacterial infection of the soft tissue. Where the symptoms of the incurvated toenail are severe, the sides of the toenail, as shown in FIG. 2, are excessively curved, thus causing pain and inflammation which may interfere with one's normal life.

Conventionally, a surgical operation has been conducted to treat the ingrown toenails and incurvated toenails. Inherently curved toenails are not straightened even after surgical treatment of the incurvated toenails, and such curved toenails present problems associated with subsequent recurrence of incurvated toenails or the incidence of pincer toenails. In the worst case, a post-surgery recurrence rate of the incurvated toenails is known to reach 80%.

U.S. Pat. No. 1,451,311 discloses an ingrown toenail corrective device formed of a pair of resilient strips. However, due to the characteristic of a slide type, the device is thick, thus making it inconvenient to use in daily life. Further, even though the part of the device in contact with the nail body should fulfill requirements in construction and material condition different from that of the part of the device in contact with the skin on both sides of the toenail, the aforesaid corrective device is made of a single elastic material, so it is difficult to meet each condition necessary for two different parts. That is, the aforesaid invention suffers from the problem in that all the parts of device are formed of a conventional elastic material, even though the part in direct contact with the nail body needs to have elasticity greater than a certain value, whereas the part in contact with sides of the toenail may suffer from processing difficulties when it is made of a highly elastic material, and selection of the material should be made in terms of sanitation because it is in direct contact with the skin. Further, since the device of the aforesaid invention is composed of the conventional elastic material, prolonged use of the device leads to permanent deformation, thus resulting in loss of effective restoring force. As a result, the device no longer exhibits corrective effects on the ingrown toenails and incurvated toenails, this consequently necessitates continuous re-placement of the device.

As another approach for corrective treatment of ingrown toenails, mention may be made of an attempt wherein a plate-like elastic material made of special steel or a synthetic resin is act into a length corresponding to a width of the toenail which is exposed to the outside and not covered with the skin, and then the thus-prepared ingrown toenail corrective device is attached to the toenail surface. However, when the special steel or elastic material having a high elastic coefficient is adhesively adhered to the toenail surface, elasticity of the material may be greater than adhesive strength, which leads to poor attachment of the device to the toenail surface. On the other hand, when the special steel or elastic material having a low elastic coefficient is used, stress greater than an elastic limit is applied to cause permanent deformation of the device. Consequently, the device no longer exhibits corrective effects on the ingrown toenails and incurvated toenails. Further, when a high-elasticity material is used, it is difficult to find a suitable adhesive for such a material. Additionally, even though there is an attempt of using the restoring force of the synthetic resin, the synthetic resin shows poor elasticity due to intrinsic nature thereof, and the part made of the resin material may be easily separated from the adhesively attached point, when it is desired to increase the thickness so as to enhance elasticity. In addition, when long-term attachment of the device is desired, this may lead to discomfort in daily life.

Korean Patent Registration No. 0485247 discloses a flat artificial toenail which has an orthopedic effect on onychocryptosis (ingrown toenail). However, this device is inconvenient for long-term use due to a large thickness. Further, since a plastic board is adhesively attached to only the exposed surface of the toenail, the corrective force of the device does not reach the region where the toenail has grown into and penetrated the nail groove, so sufficient corrective effects cannot be achieved.

Further, Japanese Patent Publication No. Hei 9-253110 discloses an ingrown nail corrective tool formed of a shape-memory alloy, but suffers from a problem in practical application thereof. Specifically, growth of a toenail after being pulled out takes about one year, so the corrective tool should be mounted onto the toenail for at least 2 to 3 months so as to control growth shape and morphology of the toenail at the early growth stage. However, due to the difference in nature between the adhesive, the metal material and the toenail, separation of the metal material from the toenail takes place easily. Further, deformation of the toenail becomes more severe toward a marginal region rather than a central part of the toenail, whereas the ingrown nail corrective tool of the aforesaid art is also designed to be adhesively attached to only the exposed surface of the toenail, so the corrective force is applied to only the central part of the toenail and therefore sufficient corrective effects cannot be achieved.

### DISCLOSURE OF INVENTION

### Technical Problem

Therefore, the present invention has been made in view of the above problems, and it is an object of the present invention to provide a corrective device for ingrown toenails and incurvated toenails, which has strong corrective force over the entire toenail, can be conveniently used, is suitable for long-term use, directs the toenail that will grow after a surgical treatment of the ingrown toenail to grow straight and is capable of straightening a curved toenail.

### Technical Solution

In accordance with an aspect of the present invention, the above and other objects can be accomplished by the provision of a corrective device for ingrown toenails and incurvated toenails, comprising a central member 13 made of a shape-memory alloy; and prefabricated toenail side-engaging parts 15, each being attached to both ends of the central member 13.

In an embodiment of the present invention, a shape-recovery temperature of the shape-memory alloy may be in a range of 25 to 30° C.

In another embodiment of the present invention, the shape-memory alloy may be a Ni—Ti-based alloy having a Ni content of 52.1 to 59.9 wt %.

In another embodiment of the present invention, a cross-sectional shape of the central member 13 may be circular or plate-like.

In another embodiment of the present invention, the toenail side-engaging part 15 may be made of stainless steel or titanium.

In another embodiment of the present invention, the toenail side-engaging part 15 may be made up of central member-connecting parts 15a, toenail bottom-support parts 15c, and connecting parts 15b for connecting between the central member-connecting part 15a and the toenail bottom-support part 15c.

In another embodiment of the present invention, the central member-connecting part 15a may have a central member-inserting part 15d therein.

In another embodiment of the present invention, a length ratio of the central member-connecting part 15a the toenail bottom-support part 15c may be in a range of 0.5:1 to 3:1.

In another embodiment of the present invention, a distance between toenail contact surfaces of the central member-connecting part 15a and the toenail bottom-support part 15c may be in a range of 0.5 to 2 mm.

In another embodiment of the present invention, a plurality of irregularities 15e may be provided on either or both of the toenail contact surface of the central member-connecting part 15a and the toenail contact surface of the toenail bottom-support part 15c.

In another embodiment of the present invention, a cross-sectional shape of the irregularities may be triangular, tetragonal or semicircular.

### Advantageous Effects

A corrective device for ingrown toenails and incurvated toenails in accordance with the present invention has strong corrective force over the entire toenail, can be conveniently used, is suitable for long-term use, directs the toenail that will grow after a surgical treatment of the ingrown toenail to grow straight and is capable of straightening a curved toenail.

### BRIEF DESCRIPTION OF THE DRAWINGS

FIG. 1 is a perspective view of a normal toenail;

FIG. 2 is a photograph showing an example of an ingrown toenail and incurvated toenail;

FIG. 3 is a perspective view showing an embodiment of a corrective device for ingrown toenails and incurvated toenails in accordance with the present invention;

FIG. 4 is a perspective view showing an embodiment of a prefabricated toenail side-engaging part in accordance with the present invention;

FIG. 5a is a side view showing another embodiment of a prefabricated toenail side-engaging part in accordance with the present invention;

FIG. 5b is a side view showing another embodiment of a prefabricated toenail side-engaging part in accordance with the present invention;

FIG. 5c is a side view showing another embodiment of a prefabricated toenail side-engaging part in accordance with the present invention; and

FIG. 6 is a photograph showing a test application of a corrective device for ingrown toenails and incurvated toenails in accordance with the present invention.

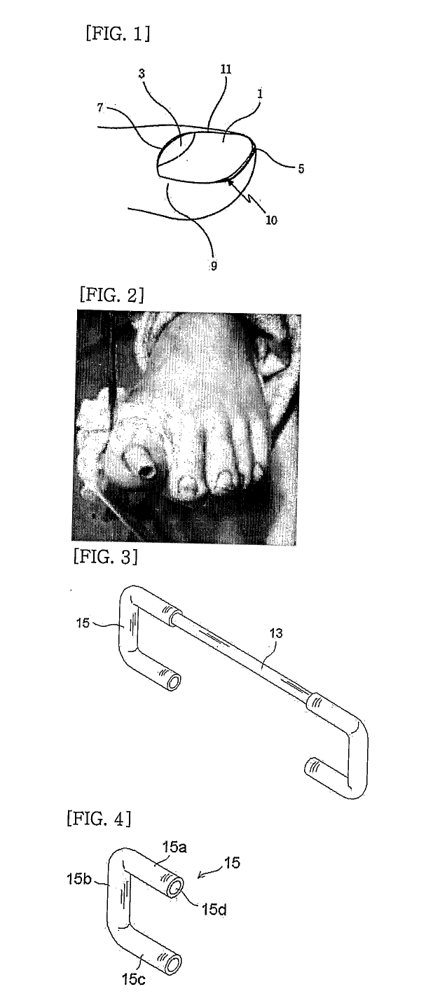

### BEST MODE FOR CARRYING OUT THE INVENTION

The present invention will now be described more fully with reference to the accompanying drawings hereinafter, in which preferred embodiments of the invention are shown. This invention may, however, be embodied in different forms and should not be construed as being limited to the embodiments set forth herein.

As shown in FIG. 3, the corrective device for ingrown toenails and incurvated toenails in accordance with the present invention comprises a central member 13 made of a shape-memory alloy; and prefabricated toenail side-engaging parts 15, each being attached to both ends of the central member 13.

The shape-memory alloy refers to an alloy that “remembers” its geometry. After a sample of shape-memory alloy has been deformed from its original crystallographic configuration, it regains its original geometry by itself during heating. The shape-memory alloy has restoring force 10 to 20-fold higher than that of iron, and exhibits smooth deformation and high anti-corrosiveness. All kinds of metals have an intrinsic elastic limit. When the metal is deformed by application of stress (stress is the applied force per unit area of a material) smaller than the elastic limit, the metal returns to the original shape if the applied stress is released. This phenomenon is called “elastic deformation”. However, when stress greater than the elastic limit is applied to the material, permanent deformation will take place which is a change in shape or dimension which does not disappear when stress is no longer applied. A degree of deformation at the point of time when permanent deformation begins to take place, i.e. deformation occurring upon application of stress greater than the elastic limit, does not exceed 1% of the original shape, and thereafter the plastic deformation that is permanent deformation takes place. Like this, an ordinary metal material keeps its deformed shape when it is deformed by application of moderate force, so it is possible to make and process desired shapes by bending or drawing the metal material. However, when the shape-memory alloy is deformed and then heated, the metal remembers its original geometry prior to deformation and then returns to the original shape. The present invention is made by taking advantage of the unique nature of the shape memory alloy that enables a deformed form to revert to a previously defined shape at a given temperature. For example, when it is desired to employ a central member which was made to remember to have a flat shape at a temperature of 27 to 28° C. corresponding to foot temperature, a shape of the central member is freely transformed to have a desired shape at a temperature of 20° C. lower than the above-specified range and then mounted onto the toenail, such that the toenail is subjected to the action of restoring force (force in direction F) generated when the member returns to the remembered shape by the action of body temperature, as shown in FIG. 6. As a result, it can be configured to ensure that the restoring force continuously acts to maintain a flat state of the toenail without re-incurvation of ingrown toenails and incurvated toenails.

A shape-recovery temperature of the shape-memory alloy may be in a range of 25 to 30° C. The above-specified range was selected taking into consideration foot temperature, because foot temperature is in a range of about 27 to 28° C., which is lower than 36.9° C. of the armpit corresponding to normal body temperature. If the shape-recovery temperature is lower than 25° C., a large decrease of the temperature is required to handle the central member made of the shape-memory alloy. On the other hand, if the shape-recovery temperature exceeds 30° C., it is probably impossible to achieve continuous maintenance of the restoring force because the central member of the shape-memory alloy is incapable of returning to a predetermined shape at normal foot temperature.

The shape-memory alloy is a nickel-, copper-, or iron-based alloy in combination with a certain metal such as zinc, aluminum, gold, silver, or the like. Examples of the shape-memory alloy that can be used in the present invention may include Ni—Ti, Cu—Zn—Ni, Cu—Al—Ni, Ag—Ni, and Au—Cd. The aforementioned shape-memory alloy generally has the attribute of superelasticity. A content of nickel in the Ni—Ti-based alloy may be in a range of 52.1% to 59.9%. Where the content of nickel is lower than 52.1% or higher than 59.9%, this may result in a difficulty to set the shape-recovery temperature of 25 to 30° C., and a difficulty to maintain elastic force suited for maintenance of the toenail in a flat form.

Even though the cross-sectional shape of the central member 13 may be circular or plate-like, there is no particular limit to the cross-sectional shape of the central member 13, so long as it is possible to achieve objects of the present invention. When the cross-sectional shape of the central member is plate-like, it is possible to increase or decrease the corrective force of the device by controlling the sectional area of the central member. Further, the central member 13 may be processed to have a proper length, depending upon a size of the toenail.

Prefabricated toenail side-engaging parts 15 are attached to both ends of the central member 13, as shown in FIG. 3. As shown in FIG. 6 when each of the toenail side-engaging parts 15 is coupled to both ends of the central member 13 and the resulting assembly is mounted onto the toenail, the upward force (force in direction F) continuously acts on the toenail along the lateral edge of such toenail by the restoring force of the shape-memory alloy, and as a reaction of that, the downward pushing force (force in direction f) of the central member 13 acts on the central part of the toenail. The downward pushing force of the central member serves as a leverage to increase F-direction force, which consequently further increases the restoring force and maintains it at a constant level. That is, it can be said that the present invention enables achievement of a three-point effect which continuously imparts the force to three points. In case of a conventional art employing an adhesive, the separation force is generated due to differences in physical properties between the adhesive and the shape-memory alloy, so it is difficult to achieve the f-direction force and it is also difficult to obtain a leverage effect. The toenail side-engaging part may be made of stainless steel or titanium which has sufficient strength and is harmless to the human body even upon long-term use. There is no particular limit to the toenail side-engaging part 15, so long as it can be engaged with lateral sides of toenail. As shown in FIG. 4, the toenail side-engaging part 15 may be made up of central member-connecting parts 15a, toenail bottom-support parts 15c, and connecting parts 15b connecting between the central member-connecting part 15a and the toenail bottom-support part 15c. The central member-connecting part 15a may include a central member-inserting part 15d therein. The toenail side-engaging parts 15 can be coupled to ends of the central member 13 by pushing ends of the central member 13 into the central member-inserting parts 15d. Alternatively, strong coupling of members may be made by applying an adhesive to ends of the central member 13 and then pushing both ends of the member 13 into the central member-inserting parts 15d, or by pushing ends of the central member 13 into the central member-inserting parts 15d and then applying a pressure to the surfaces of the central member-connecting parts 15a using a pinchcock clamp.

A length ratio of the central member-connecting part 15a: the toenail bottom-support part 15c may be in a range of 0.5:1 to 3:1. If the toenail bottom-support part 15c is shorter than the above-specified ratio, the toenail bottom-support part 15c may be easily disengaged from the toenail. On the other hand, the toenail bottom-support part 15c is longer than the above-specified ratio, the central member 13 may be easily uncoupled from the central member-connecting part 15a because the coupling between the central member-connecting part 15a and the central member 13 is not strong.

A distance between toenail contact surfaces of the central member-connecting part 15a and the toenail bottom-support part 15c may be in a range of 0.5 to 2 mm. If the distance is less than 0.5 mm, this may result in an insufficient space for binding of the toenail side-engaging part 15 to the toenail. On the other hand, if the distance exceeds 2 mm, this may result in the risk of disengagement of the toenail side-engaging part 15 from the toenail.

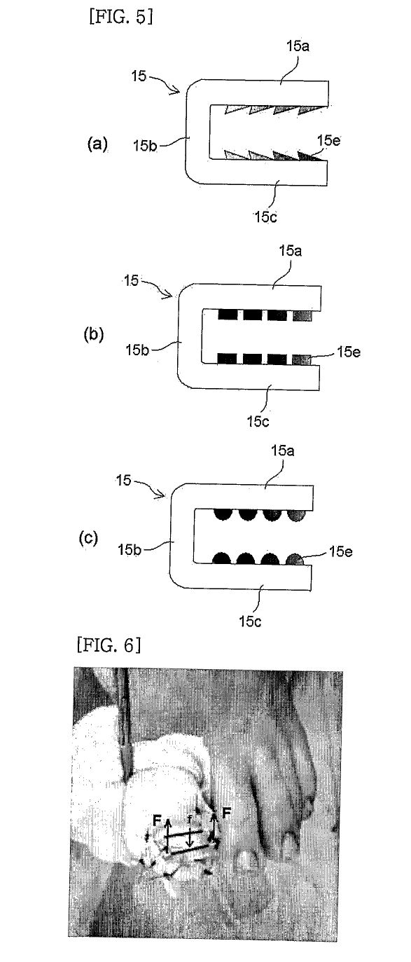

Further, a plurality of irregularities 15e may be provided on either or both of the toenail contact surface of the central member-connecting part 15a and the toenail contact surface of the toenail bottom-support part 15c The cross-sectional shape of the irregularities 15e may be triangular (FIG. 5a), tetragonal (FIG. 5b) or semicircular (FIG. 5c). By provision of the irregularities 15e, the corrective device for ingrown toenails and incurvated toenails in accordance with the present invention can be easily and firmly mounted on the toenail such that it is not disengaged from the toenail. As shown in FIG. 5a, the triangular-shaped irregularities are processed to have a saw-tooth shape, such that the device can be easily mounted on the toenail while the device cannot be easily separated from the toenail. As a result, the device can be stably used even upon long-term use. Even though it may be difficult to remove the corrective device due to strong engagement of the corrective device with the toenail as a result of the irregularities, it is possible to easily remove the corrective device from the toenail, by trimming the toenail when it grows. As described above, the corrective device for ingrown toenails and incurvated toenails in accordance with the present invention provides various advantages such as no risk of separation of the corrective device from the affected toenail during use thereof, convenient use due to use of the device in close contact with the toenail and no significant discomfort even with long-term use.

As shown in FIG. 6, it is also possible to mount one or more of the aforesaid corrective devices on one toenail, depending upon size of the toenail and when it is needed, so the corrective force of the device may be controlled to be increased or decreased as necessary. When the cross-sectional shape of the central member is plate-like, it is possible to achieve the above-mentioned purpose by controlling the sectional area of the central member.

### EXAMPLE 1

Cylindrical wire (made of stainless steel, outer diameter: 1.2 mm, inner diameter: 1.0 mm, and length: 10.0 mm, available from Cheiltech Co., Ltd., Korea) was used in preparation of toenail side-engaging parts. For this purpose, two toenail side-engaging parts were fabricated by bending the wire to have a ‘ [symbol] ’ shape, in such a manner that a central member-connecting part and a toenail bottom-support part have the same length and a distance between toenail contact surfaces of the central member-connecting part and the toenail bottom-support part is in a range of 1 mm. One end of linear wire (made of a shape-memory alloy, Ni: 55.96 wt %, O: 0.049 wt %, C: 0.031 wt %, Ti Balance; shape-recovery temperature: 27° C.; shape memory state: linear form; diameter: 1.0 mm; and length: 30 mm, available from Jinsung Industrial Co., Ltd., Korea) was inserted into one inserting part of the toenail side-engaging part, followed by fixing with a pinchcock clamp. The opposite end of the linear wire made of the shape-memory alloy, to which the cylindrical wire was not inserted, was also coupled with the toenail side-engaging part in the same manner. The corrective device for ingrown toenails and incurvated toenails as shown in FIG. 3 was assembled.

### TEST EXAMPLE 1

Toenail grown deeply into the surrounding flesh and a toenail plate where the toenail grows were partially removed, a length of the remaining toenail was measured using a template. A linear wire made of a shape-memory alloy was act into a desired length in compliance with that of the thus-measured toenail, bent in cold water, and then inserted into an inserting part of the toenail side-engaging part fabricated in Example 1, followed by fixing with a pinchcock clamp. The toenail side-engaging part coupled to the linear wire was engaged with one side of the toenail, and the toenail side-engaging part was engaged with the other side of the toenail. Then, one end of the linear wire to which the toenail side-engaging part was not coupled was pushed into a central member-inserting part of the toenail side-engaging part, followed by fixing with a pinchcock clamp. Upon maintaining such a state, the shape-memory alloy keeps its original flat state by the action of body temperature. As a result, it was possible to maintain the toenail in a regular and constant form as shown in FIG. 6, after surgical operation of an ingrown toenail.

1. A corrective device for ingrown toenails and incurvated toenails comprising:
a central member made of a shape-memory alloy; and
prefabricated toenail side-engaging parts, each being attached to both ends of the central member.

2. The corrective device according to claim 1, wherein a shape-recovery temperature of the shape-memory alloy is in the range of 25 to 30°.

3. The corrective device according to claim 1, wherein the shape-memory alloy is a Ni—Ti-based alloy having a Ni content of 52.1 to 59.9 wt %.

4. The corrective device according to claim 1, wherein a cross-sectional shape of the central member is circular or plate-like.

5. The corrective device according to claim 1, wherein the toenail side-engaging parts are made of stainless steel or titanium.

6. The corrective device according to claim 1, wherein the toenail side-engaging parts are made up of a central member-connecting part, a toenail bottom-support part, and a connecting part for connecting between the central member-connecting part and the toenail bottom-support part.

7. The corrective device according to claim 6, wherein the central member-connecting part has a central member-inserting part therein.

8. The corrective device according to claim 6, wherein a length ratio of the central member-connecting part to the toenail bottom-support part is in the range of 0.5:1 to 3:1.

9. The corrective device according to claim 6, wherein a distance between toenail contact surfaces of the central member-connecting part and the toenail bottom-support part is in the range of 0.5 to 2 mm.

10. The corrective device according to claim 6, wherein a plurality of irregularities are provided on either or both of a toenail contact surface of the central member-connecting part and a toenail contact surface of the toenail bottom-support part.

11. The corrective device according to claim 10, wherein a cross-sectional shape of the irregularities is triangular, tetragonal or semi-circular.
